(19) United States
(12) Patent Application Publication
(10) Pub. No.: US 2020/0246640 A1
(43) Pub. Date: Aug. 6, 2020

(54) SYSTEMS AND METHODS FOR USING LOW INTENSITY ULTRASONIC TRANSDUCER ON THE BRAIN

(71) Applicant: Synaptec Network, Inc., Santa Monica, CA (US)

(72) Inventors: Sheldon JORDAN, Pacific Palisades, CA (US); Alexander Sasha BYSTRISKY, Santa Monica, CA (US); Mark SCHAEFFER, Santa Monica, CA (US)

(21) Appl. No.: 16/773,912

(22) Filed: Jan. 27, 2020

Related U.S. Application Data
(60) Provisional application No. 62/799,451, filed on Jan. 31, 2019.

(51) Int. Cl. A61N 7/00; A61B 5/00; G01R 33/48; A61B 5/055; G01R 33/563

(52) U.S. Cl. CPC A61N 7/00; A61B 5/0036; G01R 33/4814; G01R 33/4806; A61B 5/0042; G01R 33/5635; A61B 5/055

(57) ABSTRACT

Methods, devices, and systems are disclosed for treating patients with ultrasound targeted at regions of the brain. An ultrasound transducer is positioned on a patient's head to target a region of the patient's brain for ultrasound therapy. An ultrasound wave is emitted at the target region of the patient's brain. At least partially concurrently, a fMRI imaging device generates real time images of activity in the patient's brain, for example by ASL protocol, BOLD protocol, or a combination thereof. Activity in the patient's brain caused by the ultrasound wave is detected, typically a distance Δ from the target region. The transducer is repositioned or reoriented to correct for Δ and better target the region, and another series of ultrasound waves are directed at the target region with improved accuracy.

This application claims priority to U.S. provisional application 62/799,451, filed Jan. 31, 2019, the disclosure of which is incorporated herein by reference.

### FIELD OF THE INVENTION

The field of the invention is methods, systems, kits, and devices related to applying ultrasonic waves to the brain.

### BACKGROUND

The background description includes information that may be useful in understanding the present invention. It is not an admission that any of the information provided herein is prior art or relevant to the presently claimed invention, or that any publication specifically or implicitly referenced is prior art.

In some cases, the most desirable way to treat an ailment is to treat the source directly. However, for conditions associated with various regions of the brain, direct treatment is typically invasive and, as such, undesirable. In such cases, indirect or noninvasive treatment of the brain is preferred. For example, “Noninvasive Focused Ultrasound for Neuromodulation: A Review” by Paul Bowary provides an overview of known uses of low intensity focused ultrasound to treat regions of the brain noninvasively. Ultrasound can be directed at targets in the brain, which is detected with functional magnetic resonance imaging (fMRI), for example the effects of ultrasound on brain tissue and network activity in other regions of the brain. Bowary further notes ultrasound can be guided via MRI in other therapies, for example FDA approved use for thalamotomy-mediated treatment of tremors.

Similarly, U.S. Pat. No. 7,283,861 to Bystritsky teaches use of low intensity focused ultrasound with fMRI to identify electrical patterns in the brain, and to modify those patterns. Ultrasound is applied to change the electrical pattern, and the fMRI is used in part to detect the changes, as well as indicate or confirm the ultrasound is directed at the targeted region of the brain. However, fMRI alone does not provide sufficient resolution to confirm the ultrasound waves are actually reaching the desired region of the brain, resulting in guess work on the part of ultrasound operators, suboptimal treatment efficacy, and potential harm to a patient.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, there remains a need for systems and methods to improve the accuracy and precision of applying ultrasound waves to targeted regions of the brain, as well as to correct and confirm such accuracy, preferably in real time.

### SUMMARY OF THE INVENTION

The inventive subject matter provides apparatus, systems, and methods to improve the accuracy and precision of targeting acoustic waves (e.g., ultrasound) to reach a desired region of a brain. An acoustic wave is applied to a targeted region of a brain by positioning a transducer (or two, or three, or more than four) to direct the acoustic wave at the targeted region of the brain. The acoustic wave is emitted at the targeted region and, preferably concurrently or substantially concurrently, a first imaging device is used to monitor activity in the brain. Viewed from another perspective, it is expected that the acoustic wave will have a detectable effect on the brain tissue it passes through, and an imaging device is used to monitor and visualize the effect in real time, preferably before, after, and as the acoustic wave is applied. It should be appreciated that detection and visualization of brain activity occurs in real time, as the acoustic wave is applied to the brain, rather than a post hoc analysis of the treatment session or cycle.

Brain activity associated with the acoustic wave is detected, typically in a region that is a distance (e.g., Δ vector, Cartesian coordinates, spherical coordinates, longitude, latitude, elevation, etc) outside of the targeted region. The transducer is then repositioned to correct for the distance, and to direct the acoustic wave closer to, preferably onto or into, the targeted region.

### BRIEF DESCRIPTION OF THE DRAWINGS

FIG. 1 depicts a flow chart of a method of the inventive subject matter.

FIG. 2 depicts a flow chart of another method of the inventive subject matter.

### DETAILED DESCRIPTION

The inventive subject matter provides apparatus, systems, and methods to improve the accuracy and precision of targeting acoustic waves (e.g., ultrasound) at desired regions of a patient's brain. An acoustic wave is applied to a targeted region of a brain by positioning a transducer (or two, or three, or more than four) to direct the acoustic wave (or waves) at the targeted region of the brain. The acoustic wave is emitted at the targeted region and, preferably concurrently or substantially overlapping, an imaging device (e.g., fMRI, ASL protocol, BOLD protocol, etc.) is used to monitor activity in the brain, preferably in real time. Viewed from another perspective, it is expected that the acoustic wave will have a detectable effect on the brain tissue it passes through, and an imaging device is used to monitor and visualize the effect in real time, preferably before, after, and as the acoustic wave is applied. It should be appreciated that detection and visualization of brain activity occurs in real time, as the acoustic wave is applied to the brain, rather than a post hoc analysis of the treatment session or cycle.

Brain activity associated with the acoustic wave is detected, typically in a region that is a distance (e.g., Δ vector, Cartesian coordinates, spherical coordinates, longitude, latitude, elevation, etc) outside of the targeted region. The transducer is then repositioned to correct for the distance, and to direct the acoustic wave closer to, preferably onto or into, the targeted region.

In some embodiments the acoustic wave is at least one of a low intensity focused ultrasound or a high intensity focused ultrasound, but it is contemplated that combinations of low and high intensity focused ultrasound having the same or different intensity or amplitude, or alternatively or in addition infrasound, can be used.

While it is contemplated that imaging devices of the inventive subject matter include all devices appropriate to detect effects of acoustic waves (e.g., ultrasound) on brain tissue, preferred embodiments contemplate a functional magnetic resonance imaging (fMRI) device (e.g., arterial spin labeling (ASL) imaging, blood oxygen level dependent (BOLD) imaging, etc). For example, some embodiments contemplate using two different fMRI devices to monitor effects of the acoustic wave on the brain, one using ASL imaging and the other using BOLD imaging, either sequentially one after the other, simultaneously, or a combination thereof. In some embodiments, a single fMRI device is used to perform both ASL imaging and BOLD imaging, either in sequence or simultaneously. In preferred embodiments, the imaging device, preferably ASL fMRI or ASL fMRI in conjunction with BOLD fMRI, is used to monitor and visualize brain activity in real time, at least partially concurrent with the application of acoustic waves to the brain. It is contemplated that real time monitoring and visualization of the interaction between acoustic waves and brain activity via ASL fMRI, BOLD fMRI, or combinations of ASL and BOLD fMRI provide substantial improvement in targeting acoustic waves toward a desired therapeutic region in the brain during treatment sessions or cycles.

While the acoustic wave can be a continuous wave or a confluence of a plurality of waves, in preferred embodiments the acoustic wave is made up of ultrasound pulses, for example pulses from more than one transducer. While it is contemplated that the targeted region of the brain is typically on the order 50 mm, 80 mm, or 100 mm deep in the brain (e.g., past hair, skin, cranium, etc), in some embodiments the targeted regions are between 8 cm and 4 cm deep in the brain, in some cases between 9 cm and 3 cm.

Transducers used to generate acoustic waves may include single element, single focus transducers, which are physically moved or angled to change the location of the targeted region within the brain. It is also contemplated that a transducer may include multiple individual acoustic emitters, allowing for changes in acoustic wave direction and focal properties to be made electronically. The means by which the acoustic energy from transducers of this type can be aimed or focused are well known in the art. By using these types of transducers, acoustic (ultrasound) energy may be directed at targeted regions of the brain without physically moving the transducer from a location (or locations if multiple transducers are used). Further the acoustic energy may be redirected by electronic means in a feedback process, based on revised targeting information. It should be appreciated a single transducer (e.g., with single acoustic emitter, multiple acoustic emitters of the same type, multiple acoustic emitters of different types, etc) can be used or more than one transducer can be used (e.g., same type of transducer, different types of transducer, etc).

Methods of treating a targeted region of a brain are further contemplated. A transducer (or two, three, or more than four) is directed to emit an acoustic wave at the targeted region. A first imaging device is used to detect a brain activity associated with the acoustic wave (e.g., change in blood flow, change in temperature, change in blood oxygen concentration, etc), and a difference between the targeted region and the detected brain activity is determined. The transducer (or one transducer of an assembly, multiple transducers in an assembly, or each transducer in an assembly) is repositioned to account for the difference between the targeted region and the detected brain activity. Once repositioned, the acoustic wave is emitted from the transducer (or one, some, most, or all of a plurality of transducers, etc) at the targeted region of the brain. It is contemplated that additional detection and repositioning be performed to improve on the accuracy and precision of affecting the targeted region of the brain with the acoustic wave.

Methods of improving treatment of a targeted region of the brain are further contemplated. A transducer is placed at a first position with a first orientation in order to direct an acoustic wave emitted from the transducer to impact the targeted region. A first acoustic wave from the transducer is emitted at the targeted region, and the resulting brain activity is monitored using an imaging device (e.g., fMRI, ASL fMRI, BOLD fMRI, whole or partial combinations thereof, etc). A change in brain activity associated with the first acoustic wave is then detected, typically such that the detected brain activity is not at or in the targeted region. The transducer is placed at a second position and a second orientation to better treat or affect the targeted region, though it is contemplated that only one of position or orientation is adjusted, or that the position or orientation of one, some, most, or all of transducers in an assembly are adjusted. A second acoustic wave is then emitted from the transducer at the targeted region, affecting brain tissue at or in the targeted region. Typically, either the first position and the second position are different from each other, or the first orientation and the second orientation are different from each other.

In some embodiments, an ultrasound transducer is placed on a patient's skull and aimed toward a targeted region of the brain, first using structural MRI to provide an approximation of the correct targeting angle of the device to reach the targeted region. This can be done either by using neuronavigation software or by using the structural T1 MRI to map the ultrasound target and determine precise distances from that target to anatomical landmarks on the skull (e.g., fiducials) which can then be used to place the ultrasound transducer in a precise location to target the desired brain region. Preferably, ASL fMRI is used to monitor blood perfusion to each region of the brain, thus detecting and visualizing changes in the flow of blood to different regions of the brain in real time. However, this can be achieved using BOLD fMRI to monitor the variation in blood oxygenation levels in different regions of the brain, which likewise detects and visualizes in real time, or combinations of ASL and BOLD methodologies. Ultrasound has been shown to cause rapid changes, particularly rapid increases, in blood perfusion in the region of ultrasound effect. Therefore, this method allows for real time spatially and temporally precise monitoring of the location and effect of ultrasound via the visualization and monitoring of changes in regional blood flow. The improvements in tracking and detecting ultrasound beam refraction through a patient's skull can also be used to further improve the initial positioning and directing of ultrasound transducers on a patient's skull, as well as to determine new optimal positions and directions.

The region of the patient's brain to be targeted by acoustic or ultrasound waves is preferably associated with a disease condition. In some embodiments, the disease condition is associated with at least one of a learning disorder, an anxiety disorder, a motor disorder, a consciousness disorder, a movement disorder, an attention disorder, a stroke, a vascular disease, dementia, progressive dementia, Alzheimer's disease, Parkinson's disease, multiple sclerosis, cancer, schizophrenia, depression, developmental disorder, substance abuse, and traumatic brain injury. However, any disease or disease condition that is pathologically associated with a region of the brain is appropriate for the contemplated methods. For example, the targeted region of the patient's brain can be the frontal lobe, parietal lobe, occipital lobe, temporal lobe, hippocampus, hypothalamus, brain stem, cerebellum amygdala, corticospinal tract, thalamus, substantia nigra, basal ganglia, a tumor, a lesion, necrotic tissue, Heschl's gyrus, Brodmann area 25, a point of injury, or any other region of interest. In some embodiments more than one region of the brain is targeted (whether simultaneously or sequentially), for example to treat more than one disease or to combat a disease associated with more than one region of the brain.

While it is contemplated the inventive subject matter is applicable to any condition (e.g., disease, disorder, characteristic, etc) associated with the brain, preferred conditions and regions of the brain include those listed in Table 1.

TABLE 1

| Condition | Region of the Brain |
|---|---|
| Alzheimer's disease: | Hippocampus and surrounding cortex |
| Parkinson's disease: | Substantia nigra and basal ganglia |
| Vascular dementia: | Diffusely throughout the brain |
| MS: | Proximal to MS lesions |
| Cancer: | Proximal to tumor and necrotic tissue |
| Schizophrenia: | Frontal lobe and Heschl's gyrus |
| Depression: | Frontal lobe and Brodmann area 25 |
| Substance abuse: | Diffusely throughout the cortex but |
| | likely not in subcortical structures |
| Traumatic Brain Injury: | Proximal to area of injury |

### Example 1

The following describes an improved concept which would eliminate many uncertainties and defects in known methods of targeting ultrasound toward regions in the brain, and allow for monitoring of the critical procedure to ensure consistency in treatment in satisfaction of regulatory requirements and repeatability using ASL. During treatment with the ultrasound transducer, the patient's brain blood perfusion is monitored and visualized in real time. Before, during and after the ultrasound pulsation, the amount of blood flow is recorded every several seconds, with exact timing differing between fMRI scanning parameters, but including at least every 0.1, 0.5, 1, 2, 3, 4, or 5 seconds. The difference in blood flow at each location in the brain from each imaging epoch to the next is then measured. The angle of displacement of the ultrasound beam through the skull and surrounding tissue is also measured to inform future targeting of the ultrasound beam outside the MR environment. The rapid, real time computing outputs images of the brain showing, with associated statistics, the regions of the brain where blood perfusion has increased or decreased. As necessary, this real time feedback of the location of ultrasound effect in the brain with respect to the targeted region is used to adjust placement and direction of the ultrasound transducer before the patient receives the full or subsequent dose of ultrasound. At the end of treatment, or at an appropriate interstitial period, the total amount of change in blood flow throughout the brain associated with the ultrasound treatment is measured to track ultrasound treatment. This allows for assessment of changes in not only blood perfusion but functional connectivity between different brain regions as a function of ultrasound treatment.

### Example 2

The following describes an improved concept which would eliminate many uncertainties and defects in known methods of targeting ultrasound toward regions in the brain, and allow for monitoring of the critical procedure to ensure consistency in treatment in satisfaction of regulatory requirements and repeatability using BOLD. During treatment of patient with an ultrasound transducer, the patient's brain blood oxygenation is monitored and visualized in real time. Before, during and after the ultrasound pulsation, the amount of oxygenated blood is recorded every several seconds, with exact timing differing between fMRI scanning parameters, but including at least every 0.1, 0.5, 1, 2, 3, 4, or 5 seconds. The difference in blood oxygenation at each location in the brain from each imaging epoch to the next is then measured. The difference in blood oxygenation is compared between epochs when the ultrasound transducer is on versus off. The angle of displacement of the ultrasound beam through the skull and surrounding tissue is also measured to inform future targeting of the ultrasound transducer outside the MR environment. The rapid, real time computing outputs images of the brain showing, with associated statistics, the regions of the brain where blood oxygenation has increased or decreased as a function of the ultrasound treatment. As necessary, with this real time feedback of the location of ultrasound effect, adjustments in placement and direction of the ultrasound transducer are made before the patient receives the full or subsequent dose of ultrasound. At the end of treatment, the total amount of change in blood oxygenation throughout the brain associated with the ultrasound treatment is measured to track treatment. This allows for assessment of changes in not only oxygenation but functional connectivity between different brain regions as a function of ultrasound treatment.

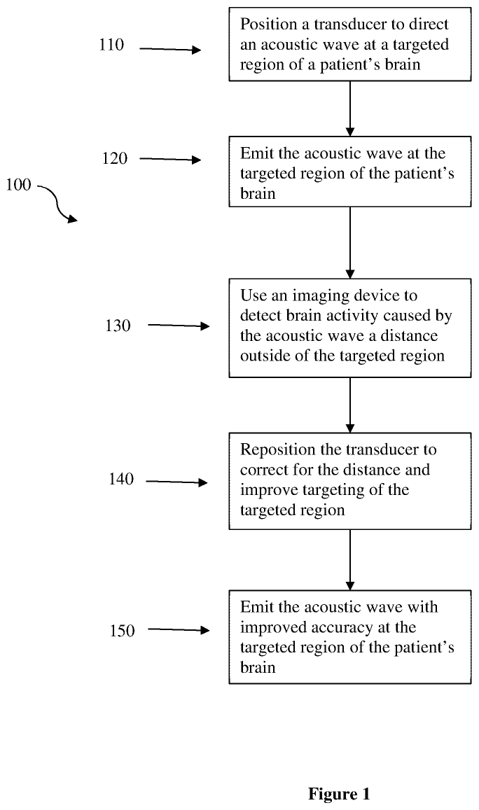

FIG. 1 depicts flowchart 100 for methods of the inventive subject matter for treating a patient. In step 110, a transducer (e.g., ultrasound transducer) is placed on or near the patient and oriented to emit an acoustic wave (e.g., ultrasound pulse) toward a targeted region of the patient's brain for therapy. In step 120, an acoustic wave is emitted toward the targeted region of the patient's brain. It is contemplated that the acoustic wave can be emitted continuously, in pulses, with varying frequency, amplitude, or duration, etc. The acoustic wave has a detectable effect on the brain that can be detected and imaged in real time, for example by fMRI using an ASL or BOLD protocol.

In step 130, an imaging device (e.g., fMRI, ASL protocol, BOLD protocol, combination thereof, etc) is used to detect brain activity caused by the acoustic wave in the patient's brain in realtime. It is contemplated that in some cases the brain activity will be in the targeted region of the brain, requiring no further adjustment. However, the imaging device will also detect when the brain activity caused by the acoustic wave is outside of the targeted region. In such cases, the distance of the activated region and the targeted region is determined based on the imaging device data, preferably in realtime. In step 140, this distance is used to reposition (e.g., translate, rotate, etc.) to correct for the distance and improve targeting of the targeted region. With corrected targeting, step 150 emits an acoustic wave with improved accuracy at the targeted region of the patient's brain, preferably causing a detectable change in brain activity in the targeted region.

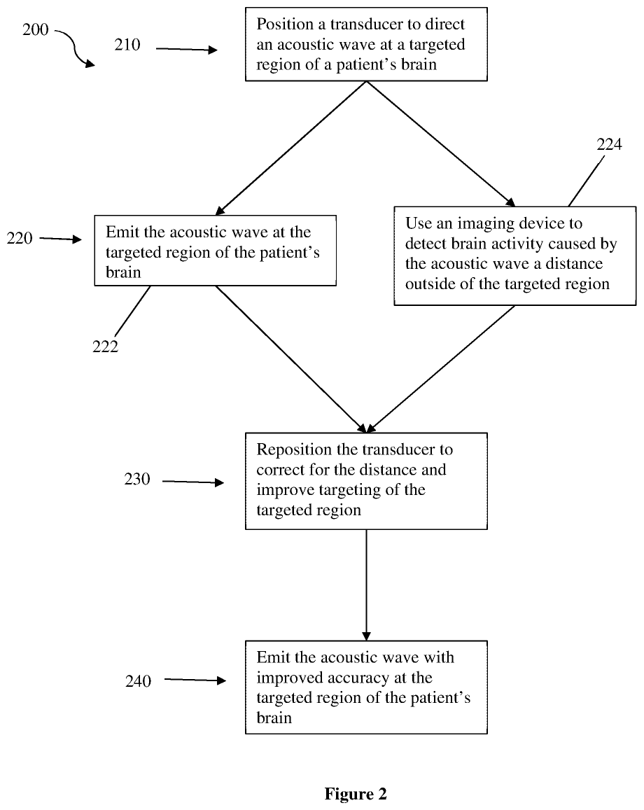

FIG. 2 depicts a flowchart 200 for methods of the inventive subject matter, similar to FIG. 1. However, in FIG. 2, step 220 comprises the simultaneous or substantially overlapping substeps 222 and 224. In substep 222, the acoustic wave is emitted at the targeted region of the patient's brain. Simultaneously or at least partially overlapping (e.g., during step 222, after step 222 begins, before step 222 begins and continuing with step 222, etc.), step 224 uses an imaging device to detect brain activity caused by the acoustic wave. It is contemplated that simultaneous use of the transducer to activity regions of the patient's brain along with realtime imaging of changes in the patient's brain activity permits operators to adjust and improve targeting of the targeted region in realtime.

Various objects, features, aspects, and advantages of the inventive subject matter will become more apparent from the following detailed description of preferred embodiments, along with the accompanying drawing figures in which like numerals represent like components.

The following description includes information that may be useful in understanding the present invention. It is not an admission that any of the information provided herein is prior art, necessary, or relevant to the presently claimed invention, or that any publication specifically or implicitly referenced is prior art.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of applying an acoustic wave to a targeted region of a brain, comprising:
positioning a transducer to direct the acoustic wave at the targeted region;
concurrently (i) emitting the acoustic wave at the targeted region and (ii) using a first imaging device to monitor activity in the brain;
detecting brain activity associated with the acoustic wave a distance outside of the targeted region; and
repositioning the transducer to correct for the distance and direct the acoustic wave at the targeted region.

2. The method of claim 1, wherein the acoustic wave is at least one of a low intensity focused ultrasound or a high intensity focused ultrasound.

3. The method of claim 1, wherein the first imaging device is a functional magnetic resonance imaging (fMRI) device.

4. The method of claim 3, wherein the fMRI device visualizes activity in the brain in real time.

5. The method of claim 3, wherein the fMRI device uses arterial spin labeling (ASL) imaging.

6. The method of claim 5, further comprising the step of using a second imaging device to monitor activity in the brain.

7. The method of claim 6, wherein the second imaging device is a fMRI device using blood oxygen level dependent (BOLD) imaging.

8. The method of claim 6, wherein the second imaging device visualizes activity in the brain in real time.

9. The method of claim 1, wherein the acoustic wave comprises a plurality of ultrasound pulses.

10. The method of claim 1, wherein the targeted region is between 8 cm and 4 cm deep in the brain.

11. A method of treating a targeted region of a brain, comprising:
directing a transducer to emit an acoustic wave at the targeted region;
using a first imaging device to detect a brain activity associated with the acoustic wave;
determining a difference between the targeted region and the detected brain activity;
redirecting the transducer to account for the difference between the targeted region and the detected brain activity and;
emitting the acoustic wave at the targeted region.

12. The method of claim 11, wherein the acoustic wave is at least one of a low intensity focused ultrasound or a high intensity focused ultrasound.

13. The method of claim 11, wherein the first imaging device is a fMRI device.

14. The method of claim 13, wherein the fMRI device visualizes activity in the brain in real time.

15. The method of claim 13, wherein the fMRI device uses ASL imaging.

16. The method of claim 15, further comprising the step of using a second imaging device to monitor activity in the brain.

17. The method of claim 16, wherein the second imaging device is a fMRI device using BOLD imaging.

18. The method of claim 16, wherein the second imaging device is visualizes activity in the brain in real time.

19. A method of improving treatment of a targeted region of the brain, comprising:
placing a transducer at a first position and a first orientation to direct an acoustic wave at the targeted region;
emitting a first acoustic wave from the transducer at the targeted region and monitoring brain activity using an imaging device;
detecting a change in brain activity associated with the first acoustic wave, wherein the detected brain activity is not at the targeted region;
placing the transducer at a second position and a second orientation to better treat the targeted region; and
emitting a second acoustic wave from the transducer at the targeted region.

20. The method of claim 19, wherein at least one of the first position is different from the second position, or the first orientation is different than the second orientation.

21. The method of claim 19, wherein the first acoustic wave is a low intensity focused ultrasound comprising a plurality of pulses.

22. The method of claim 19, wherein the imaging device is a fMRI using ASL imaging to visualize brain activity in real time.
